(19) United States
(12) Patent Application Publication
(10) Pub. No.: US 2007/0219183 A1
(43) Pub. Date: Sep. 20, 2007

(54) Dihydropyrazolo[1,5-A]pyrimidine and dihydroimidazo[1,5-A]pyrimidine derivatives and methods of use thereof

(75) Inventors: Jeremy Ian Levin, New City, NY (US); Zhong Li, Congers, NY (US); Dennis Powell, Cortlandt Manor, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/724,489

(22) Filed: Mar. 15, 2007

Related U.S. Application Data
(60) Provisional application No. 60/783,255, filed on Mar. 17, 2006.

(51) Int. Cl. A61K 31/554; A61K 31/553; A61K 31/551; A61K 31/541; A61K 31/5377; A61K 31/519; C07D 487/04

(52) U.S. Cl. 514/211.15; 514/259.1; 514/259.3; 514/234.2; 514/252.16; 514/227.8; 514/218; 514/217.06; 544/281; 544/114; 544/60; 540/575; 540/544; 540/600

(57) ABSTRACT

The present invention relates to dihydropyrazolo[1,5-a]pyrimidine and dihydroimidazo[1,5-a]pyrimidine derivatives, compositions comprising an effective amount of a dihydropyrazolo[1,5-a]pyrimidine or a dihydroimidazo[1,5-a]pyrimidine derivative and methods for treating or preventing cancer, comprising administering to a subject in need thereof an effective amount of a dihydropyrazolo[1,5-a]pyrimidine or a dihydroimidazo[1,5-a]pyrimidine derivative.

This application claims priority from copending U.S. provisional application No. 60/783,255, filed Mar. 17, 2006, the entire disclosure of which is hereby incorporated by reference.

The present invention relates to dihydropyrazolo[1,5-a]pyrimidine and dihydroimidazo[1,5-a]pyrimidine derivatives, compositions comprising an effective amount of a dihydropyrazolo[1,5-a]pyrimidine or a dihydroimidazo[1,5-a]pyrimidine derivative and methods for treating or preventing cancer, comprising administering to a subject in need thereof an effective amount of a dihydropyrazolo[1,5-a]pyrimidine or a dihydroimidazo[1,5-a]pyrimidine derivative.

Cancer is second only to cardiovascular disease as a cause of death in the United States. The American Cancer Society estimated that in 2004, there were 1.6 million new cases of cancer and 655,000 cancer-related deaths. There are currently over 10 million living Americans who have been diagnosed with cancer and the NIH estimates the direct medical costs of cancer as over $100 billion per year with an additional $100 billion in indirect costs due to lost productivity—the largest such costs of any major disease.

Cancer is a process by which the controlling mechanisms that regulate cell growth and differentiation are impaired, resulting in a failure to control cell turnover and growth. This lack of control causes a tumor to grow progressively, enlarging and occupying space in vital areas of the body. If the tumor invades surrounding tissue and is transported to distant sites, death of the individual can result.

The selective killing of cancer cells, while minimizing deleterious effects on normal cells, is a desired goal in cancer therapy. Modalities commonly used in the treatment of cancer include chemotherapy, radiation therapy, surgery and biological therapy (a broad category that includes gene-, protein- or cell-based treatments and immunotherapy). Despite the availability of a variety of anticancer agents, traditional chemotherapy has drawbacks. Many anticancer agents are toxic, and chemotherapy can cause significant, and often dangerous, side effects, including severe nausea, bone marrow depression, liver, heart and kidney damage, and immunosuppression. Since it is difficult to predict the pattern of sensitivity of a neoplastic cell population to anticancer drugs, it is common to use multi-drug regimens.

Despite the significant research efforts and resources which have been directed towards the development of novel anticancer agents and improved methods for treating cancer there remains a need in the art for novel compounds, compositions, and methods that are useful for treating cancer.

### SUMMARY OF THE INVENTION

In one aspect the invention provides a compound of Formula (I):

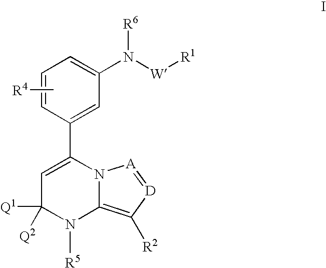

or a pharmaceutically acceptable salt thereof,

wherein

A is C—R³ or N;

D is N or C—R³ provided when D is N, A is C—R³;

R¹ is phenyl, naphthyl, or a 5-10 membered mono- or bicyclic heteroaryl ring containing 1-3 heteroatoms selected from N, O or S, wherein the phenyl, naphthyl and heteroaryl rings are optionally substituted with one to four substituents selected from the group consisting of -J, —NO₂, —CN, —N₃, —CHO, —CF₃, —OCF₃, —R⁷, —OR⁷, —S(O)ₘR⁷, —NR⁷R¹², —NR¹³S(O)ₘR⁷, —OR⁹OR⁷, —OR⁹NR⁷R¹², —N(R¹¹)R⁹OR⁷, —N(R¹³)R⁹NR⁷R¹⁴, —NR¹³C(O)R⁷, —C(O)R⁷, —C(O)OR⁷, —C(O)NR⁷R¹², —OC(O)R⁷, —OC(O)OR⁷, —OC(O)NR⁷R¹², NR¹¹C(O)R⁷, —NR¹¹C(O)OR⁷, —NR¹¹C(O)NR⁷R¹², —R⁸OR⁷, —R⁸NR⁷R¹², —R⁸S(O)ₘR⁷, —R⁸C(O)R⁷, —R⁸C(O)OR⁷, —R⁸C(O)NR⁷R¹², —R⁸OC(O)R⁷, —R⁸OC(O)OR⁷, —R⁸OC(O)NR⁷R¹², —R⁸NR¹¹C(O)R⁷, —R⁸NR¹¹C(O)OR⁷, —R⁸NR¹¹C(O)NR⁷R¹², —YR⁸R¹⁰, —YR⁸NR⁷R¹² or —YR¹⁰;

R² and R³ are independently selected from R⁷, J, —C(O)OR⁷, —C(O)NR⁷R¹², —NR⁶C(O)R⁷, —CN, 5-7 membered heterocyclic ring, 5-10 membered heteroaryl ring containing 1-3 heteroatoms selected from N, O or S, or aryl ring, wherein the R⁷ groups, heterocyclic, heteroaryl and aryl rings can be optionally substituted with one to four substituents selected from the group consisting of -J, —NO₂, —CN, —N₃, —CHO, —CF₃, —OCF₃, —R¹³, —OR¹³, —S(O)ₘR¹³, —NR¹³R¹⁴, —NR¹¹S(O)ₘR, —OR⁹OR¹³, —R⁹NR¹³R¹², —N(R¹¹)R⁹OR¹³, —N(R¹¹)R⁹NR¹³R¹², —NR¹¹C(O)R¹³, —C(O)R¹³, —C(O)OR¹³, —C(O)NR¹³R¹², —OC(O)R¹³, —OC(O)OR¹³, —OC(O)NR¹³R¹², NR¹¹C(O)R¹³, —NR¹¹C(O)OR¹³, —NR¹¹C(O)NR¹³R¹², —R⁸OR¹³, —R⁸NR¹³R¹², —R⁸S(O)ₘR¹³, —R⁸C(O)R¹³, —R⁸C(O)OR¹³, —R⁸C(O)NR¹³R¹², —R⁸OC(O)R¹³, —R⁸OC(O)OR¹³, —R⁸OC(O)NR¹³R¹²,R⁸NR¹¹C(O)R¹³, —R⁸NR¹¹C(O)OR¹³or —R⁸NR¹¹C(O)NR¹³R¹² or YR¹⁰;

R⁴ is H, -J, —NO₂, —CN, —N₃, —CHO, —CF₃, —OCF₃, —R⁷, —OR⁷, —S(O)ₘR⁷, —NR⁷R¹², —NR¹¹S(O)ₘR⁷, —OR⁹OR⁷, —OR⁹NR⁷R¹², —N(R¹¹)R⁹OR⁷, —N(R¹¹)R⁹OR⁷, —N(R¹¹)R⁹NR⁷R¹², —NR¹¹C(O)R⁷, —C(O)R⁷, —C(O)OR⁷, —C(O)NR⁷R¹², —OC(O)R⁷, —OC(O)OR⁷, —OC(O)NR⁷R¹², NR¹¹C(O)R⁷, —NR¹¹C(O)OR⁷, —NR¹¹C(O)NR⁷R¹², —R⁸OR⁷, —R⁸NR⁷R¹², —R⁸S(O)ₘR⁷, —R⁸C(O)R⁷, —R⁸C(O)OR⁷, —R⁸C(O)NR⁷R¹², —R⁸OC(O)R⁷, —R⁸OC(O)OR⁷, —R⁸OC(O)NR⁷R¹², —R⁸NR¹¹C(O)R⁷, —R⁸NR¹¹C(O)OR⁷ or R⁸NR¹¹C(O)NR⁷R¹² or YR¹⁰;

R⁵ is H, —CHO, —R⁷, —S(O)ₘR⁷, —C(O)R⁷, —C(O)OR⁷, —C(O)NR⁷R¹², —R⁸OR⁷, —R⁸NR⁷R¹², —R⁸S(O)ₘR⁷, —R⁸C(O)R⁷, —R⁸C(O)OR⁷, —R⁸C(O)NR⁷R¹², —R⁸OC(O)R⁷, R⁸OC(O)OR⁷, —R⁸OC(O)N R⁷R¹², —R⁸N R¹¹C(O)R⁷, —R⁸NR¹¹C(O)OR⁷ or —R⁸NR¹¹C(O)NR⁷R¹² or YR¹⁰;

R⁶is H;

R⁷ is H, alkyl of 1-6 carbon atoms, branched alkyl of 1-8 carbon atoms, cis-alkenyl of 2-6 carbon atoms, a trans-alkenyl of 2-6 carbon atoms, or an alkynyl of 2-6 carbon atoms, each said alkyl, alkenyl, and alkynyl being optionally substituted with -J, —NO₂, —CN, —N₃, —CHO, —CF₃, —OCF₃, —R¹³, —OR¹³, —S(O)ₘR¹³, —NR¹³R¹⁴, —NR¹³S(O)ₘR¹⁴, —OR⁹OR¹³, —OR⁹NR¹³R¹⁴, —N(R¹³)R⁹OR¹⁴, —N(R¹³)R⁹NR¹⁴R¹⁵, —NR¹³C(O)R¹⁴, —C(O)R¹³, —C(O)OR¹³, —C(O)N R¹³R¹⁴, —OC(O)R¹³, —OC(O)OR¹³, —OC(O)NR¹³R¹⁴, NR¹³C(O)R¹⁴, —NR¹³C(O)OR¹⁴, —NR¹³C(O)NR¹⁴R⁵, —R⁸OR¹³, —R⁸NR¹³R¹⁴, —R⁸S(O)ₘR¹³, —R⁸C(O)R¹³, —R⁸C(O)OR¹³, —R⁸C(O)NR¹³R¹⁴, —R⁸OC(O)R¹³, —R⁸OC(O)OR¹³, —R⁸OC(O)NR¹³R¹⁴, —R⁸NR¹³C(O)R¹⁴, —R⁸NR¹³C(O)OR¹⁴, —R⁸NR¹³C(O)NR¹⁴R¹⁵ or —YR¹⁰;

R⁸ is a divalent group comprising alkyl of 1-6 carbon atoms, alkenyl of 2-6 carbon atoms, and alkynyl of 2-6 carbon atoms;

R⁹ is a divalent alkyl group of 2-6 carbon atoms;

R¹⁰ is a cycloalkyl ring of 3-10 carbons; or a bicycloalkyl ring of 3-10 carbons; or aryl; or heterocyclic or a heteroaryl ring containing 1-3 heteroatoms selected from N, O or S; or a heteroaryl fused to one to three aryl or heteroaryl rings; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R⁷, —OR⁷, —S(O)ₘR⁷, —NR⁷R¹², —NR¹¹S(O)ₘR⁷, —OR⁹OR⁷, —OR⁹NR⁷R¹², —N(R¹¹)R⁹OR⁷, —N(R¹¹)R⁹NR⁷R¹², —NR¹¹C(O)R⁷, —C(O)R⁷, —C(O)OR⁷, —C(O)N R⁷R¹², —OC(O)R⁷—, —OC(O)OR⁷, —OC(O)NR⁷R¹², —NR¹¹C(O)R⁷, —NR¹¹C(O)OR⁷, —NR¹¹C(O)NR⁷R¹², —R⁸OR⁷, R⁸NR⁷R¹², —R⁸S(O)ₘR⁷, —R⁸C(O)R⁷, 13 R⁸C(O)OR⁷, —R⁸C(O)N R⁷R¹², —R⁸C(O)R⁷, —R⁸C(O)OR⁷, —R⁸C(O)NR⁷R¹², —R⁸OC(O)R⁷, —R⁸OC(O)OR⁷, —R⁸OC(O)NR⁷R¹², —R⁸NR¹¹C(O)R⁷, —R⁸NR¹¹C(O)OR⁷, or —R⁸NR¹¹C(O)NR⁷R¹²;

R¹¹ is H, alkyl of 1-6 carbon atoms, branched alkyl of 1-8 carbon atoms, cis-alkenyl of 2-6 carbon atoms, a trans- alkenyl of 2-6 carbon atoms, or an alkynyl of 2-6 carbon atoms;

R¹² is H, alkyl of 1-6 carbon atoms, branched alkyl of 1-8 carbon atoms, cis-alkenyl of 2-6 carbon atoms, a trans-alkenyl of 2-6 carbon atoms, or an alkynyl of 2-6 carbon atoms;

R⁷ and R¹² together with the N to which they are attached may join to form a 3 to 8 membered ring;

R¹³, R¹⁴, and R¹⁵ are independently selected from the group of H, alkyl of 1-6 carbon atoms, branched alkyl of 1-8 carbon atoms, cis-alkenyl of 2-6 carbon atoms, a trans-alkenyl of 2-6 carbon atoms, and an alkynyl of 2-6 carbon atoms;

J is fluoro, chloro, bromo, and iodo.

m is an integer of 0-2;

W′ is —C(O);

Y is a bond, a divalent alkyl group of 1-6 carbon atoms, —NH—, —O—, —NR⁷—, —C═C—, cis- CH═CH—, or trans- —CH═CH—; and

Q¹ and Q² are hydrogen or when taken together with the carbon to which they are attached may form a carbonyl (C═O) group.

In an embodiment of the invention A is N and D is C—R₃;

In a further embodiment of the invention A is C—R³ and D is N;

In one embodiment of the invention Q₁ and Q2 are H;

In an embodiment of the invention Q₁ and Q2 taken together with the carbon to which they are attached forms a carbonyl;

In another embodiment of the invention R₁ is 3-trifluoromethylphenyl;

Another embodiment of the invention is a mixture comprising a compound of formula 1 or a pharmaceutically acceptable salt thereof and an impurity. An impurity is any other chemical or biological entity, such as a different compound, stereoisomer, salt, intermediate, or pollutant of any sort. An impurity may be present in the mixture in an amount greater than the compound itself, but typically is present in an amount less than the desired compound. In another aspect of the invention the impurity may be present in an amount of less than 10% of the amount of the compound. In another aspect of the invention the impurity may be present in an amount of less than 10% of the mixture.

In an embodiment of the invention compounds or pharmaceutically acceptable salts thereof are:
N-[3-(4,5-dihydropyrazolo[1,5-a]pyrimidin-7-yl)phenyl]-3-(trifluoromethyl)benzamide;
N-{3-[4-(methylsulfonyl)-4,5-dihydropyrazolo[1,5-a]pyrimidin-7-yl]phenyl}-3-(trifluoromethyl)benzamide;
methyl 7-(3-{[3-(trifluoromethyl)benzoyl]amino}phenyl)pyrazolo[1,5-a]pyrimidine-4(5H)-carboxylate;
ethyl 7-(3-{[3-(trifluoromethyl)benzoyl]amino}phenyl)pyrazolo[1,5-a]pyrimidine-4(5H)-carboxylate;
N-isopropyl-7-(3-{[3-(trifluoromethyl)benzoyl]amino}phenyl)pyrazolo[1,5-a]pyrimidine-4(5H)-carboxamide;
tert-butyl 7-(3-{[3-(trifluoromethyl)benzoyl]amino}phenyl)pyrazolo[1,5-a]pyrimidine-4(5H)-carboxylate;
N-[3-(4-but-2-yn-1-yl-4,5-dihydropyrazolo[1,5-a]pyrimidin-7-yl)phenyl]-3-(trifluoromethyl)benzamide;
ethyl 7-(3-{[3-(trifluoromethyl)benzoyl]amino}phenyl)-4,5-dihydropyrazolo[1,5-a]pyrimidine-3-carboxylate;
N-ethyl-7-(3-{[3-(trifluoromethyl)benzoyl]amino}phenyl)pyrazolo[1,5-a]pyrimidine-4(5H)-carboxamide;
N-[3-(4-acetyl-4,5-dihydropyrazolo[1,5-a]pyrimidin-7-yl)phenyl]-3-(trifluoromethyl)benzamide;
ethyl 4-acetyl-7-(3-{[3-(trifluoromethyl)benzoyl]amino}phenyl)-4,5-dihydropyrazolo[1,5-a]pyrimidine-3-carboxylate;
tert-butyl 7-(3-{(tert-butoxycarbonyl)[3-(trifluoromethyl)benzoyl]amino}phenyl)pyrazolo[1,5-a]pyrimidine-4(5H)-carboxylate 4(5H)-carboxylate;
3-ethyl 4-methyl 7-(3-{[3-(trifluoromethyl)benzoyl]amino}phenyl)pyrazolo[1,5-a]pyrimidine-3,4(5H)-dicarboxylate;
tert-butyl {2-oxo-2-[7-(3-{[3-(trifluoromethyl)benzoyl]amino}phenyl)pyrazolo[1,5-a]pyrimidin-4(5H)-yl]ethyl}carbamate;
N-[3-(4-glycyl-4,5-dihydropyrazolo[1,5-a]pyrimidin-7-yl)phenyl]-3-(trifluoromethyl)benzamide;
N-{3-[4-(N-acetylglycyl)-4,5-dihydropyrazolo[1,5-a]pyrimidin-7-yl]phenyl}-3-(trifluoromethyl)benzamide;
tert-butyl {3-oxo-3-[7-(3-{[3-(trifluoromethyl)benzoyl]amino}phenyl)pyrazolo[1,5-a]pyrimidin-4(5H)-yl]propyl}carbamate;
N-[3-(4-β-alanyl-4,5-dihydropyrazolo[1,5-a]pyrimidin-7-yl)phenyl]-3-(trifluoromethyl)benzamide;
N-{3-[4-(N-acetyl-β-alanyl)-4,5-dihydropyrazolo[1,5-a]pyrimidin-7-yl]phenyl}-3-(trifluoromethyl)benzamide; or
N-[4-chloro-3-(4,5-dihydropyrazolo[1,5-a]pyrimidin-7-yl)phenyl]-3-(trifluoromethyl)benzamide.

The following definitions are used in connection with the dihydropyrazolo[1,5-a]pyrimidine and dihydroimidazo[1,5-a]pyrimidine derivatives of the invention.

Aryl means an organic radical derived from an aromatic hydrocarbon by the removal of one atom. Examples of aryl include phenyl and napthyl. The aryl group may be optionally substituted.

Carrier shall encompass pharmaceutically acceptable carriers, excipients, and diluents.

Heteroaryl means the class of heterocyclyl groups derived from heteroarenes by removal of one hydrogen atom from a ring atom. Examples of heteroaryl include 2-pyridyl or indol-1-yl. The heteroaryl group may be optionally substituted.

Heterocyclic, heterocycle, or heterocyclyl means a cyclic organic radical wherein at least one of the ring atoms is N, O, or S. The heterocyclic, heterocycle or heterocyclyl group may be optionally substituted. The heterocyclic, heterocycle or heterocyclyl group may contain one of more fused rings.

The compounds of this invention may be prepared from: (a) commercially available starting materials (b) known starting materials which may be prepared as described in literature procedures or (c) new intermediates described in the schemes and experimental procedures herein.

Reactions are performed in a solvent appropriate to the reagents and materials employed and suitable for the transformation being effected. It is understood by those skilled in the art of organic synthesis that the various functionalities present on the molecule must be consistent with the chemical transformation proposed. This may necessitate a certain order of synthetic steps.

Compounds of the present invention may be prepared as illustrated in the examples and in following reaction schemes 1 to 6.

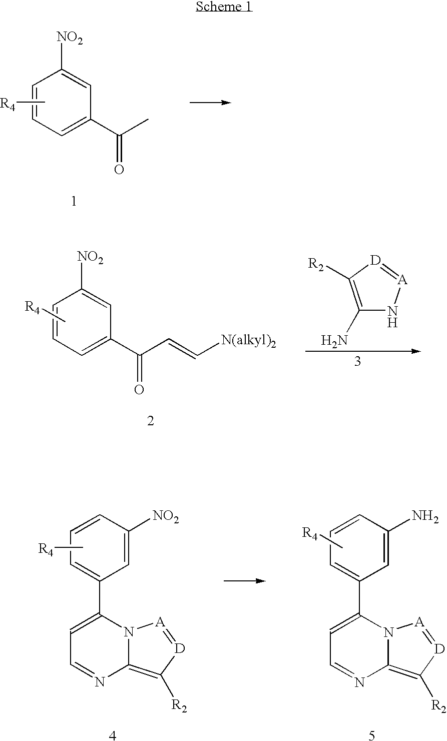

Referring to Scheme 1, the reaction of 3-nitroacetophenones, 1, optionally substituted with R₄, with acetals of N,Ndialkylformamides or acetals of N,N-dialkylacetamide can be performed with or without an inert solvent to give 3-dialkylamino-1-(aryl or heteroaryl)-2-propen-1-ones 2. The reaction of the substituted amine 3 as a 3-aminopyrazole where A is N and D is (C—R₃) and where R₂ and R₃ are herein before defined, or a 1H-imidazol-5-amine where A is C and D is N in a weak acid such as glacial acetic acid or in an inert solvent such as toluene, acetonitrile or dimethoxyethane, at reflux temperature for several hours, produces nitro compounds 4. 3-Amino-4-pyrazoles are disclosed in U.S. Pat. Nos. 4,236,005; 4,281,000; 4,521,422; 4,626,538; 4,654347; and 4,900,836 and an appropriately substituted. 3-Dialkylamino-1-(aryl or heteroaryl)-2-propen-1-one and in particular 3-dialkylamino-1-phenyl-2-propen-1-ones are disclosed in U.S. Pat. Nos. 4,178,449 and 4,236,005. Substituted 3-dimethylamino-1-(3-heteroaryl)-2-propen-1-ones are disclosed in U.S. Pat. Nos. 4,281,000 and 4,521,422. Pyrazolo[1,5-a]pyrimidines are prepared by condensation of 3-aminopyrazoles and substituted 3-aminopyrazoles with 1,3-dicarbonyl compounds as described in J. Med. Chem., 18, 645 (1974); J. Med. Chem. 18, 460 (1975); J. Med. Chem., 20, 386 (1977); Synthesis, 673 (1982).

The reduction of nitro compound, 4 with reducing agents such as Fe, SnC1₂-xH₂O, catalytic hydrogenation and the like, gives aniline 5.

As described in Scheme 2, aniline 5 is reacted with acylating agents such as an acyl chloride 6 or an carboxylic acid anhydride prepared from carboxylic acid 7, in the presence of a organic base such as pyridine, triethylamine, N-methylmorpholine, 4-dimethylaminopyridine and the like, to give amide compounds, 8. Alternatively, amide 8 can be prepared from the reaction of aniline 5 with a carbamate intermediate generated in situ by treating carboxylic acid 7 with an alkyl chloroformate in the presence of a organic base such as pyridine, triethylamine, N-methylmorpholine, 4-dimethylaminopyridine and the like. Amide 8 can also be prepared by reacting carboxylic acid 7 in the presence of coupling reagents like 1-(3-Dimethylaminopropyl)-3-ethylcarbodiimide hydrochloride(EDCI), 1,3-dicyclohexylcarbodiimide (DCC), 1-hydroxybenzotriazole hydrate (HOBT), O-(benzotriazol-1-yl)-N,N,N′,N′-tetramethyluronium hexafluorophosphate(HBTU) and the like, in the presence of organic base such as pyridine, triethylamine, N-methylmorpholine, 4-dimethylaminopyridine and the like followed by further reaction with aniline 5. Alternatively, amide 8 are also be prepared by acylation of 3-amino acetophenone 9 followed by reaction of the resulting amide 10 with a DMF dialkyl acetal 3 as described in Scheme 1, to provide intermediate enaminone 10 followed by reaction with the desired 3-aminopyrazole 3, described in Scheme 1, to give amide 8.

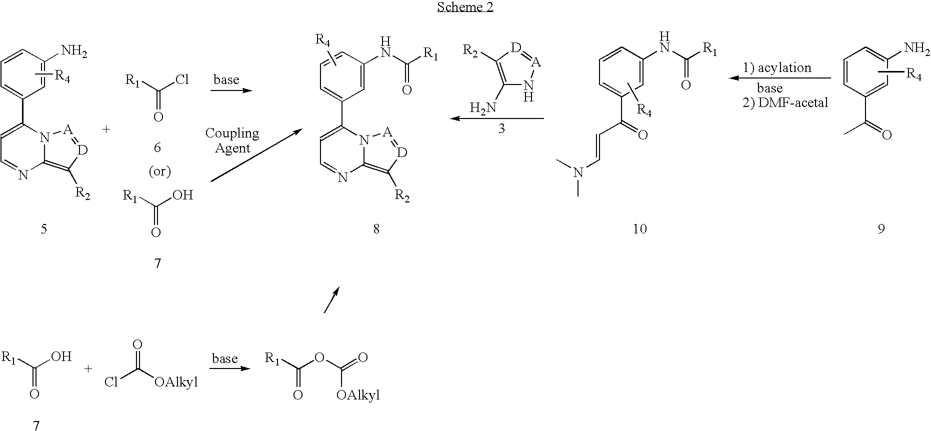

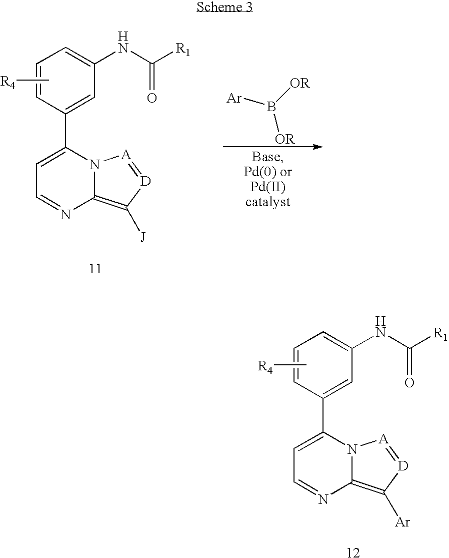

As shown in scheme 3, certain dihydropyrazolo[1,5-a]pyrimidine and dihydroimidazo[1,5-a]pyrimidine derivatives 12, where Ar is an optionally substituted aryl, heteroaryl, or heterocyclic ring and where R₁, R₃, and R₄ are as defined herein above can be synthesized using starting material 11, where J is bromo or iodo which can be prepared by the methodology outlined in Schemes 1 and 2, or by the reaction of amide 8 of Scheme 2 with an N-halosuccinimide. Substituted ArB(OR)₂ are either commercially available as boronic acids or esters, or can for example be synthesized from the corresponding aryl and heteroaryl halides by methodology described in: Hamel, E. et al. Journal of Medicinal Chemistry. 1992, 35 1058-1067 and Berger, D. et al. Bioorganic & Medicinal Chemistry Letters 2002, 12, 2989-2992. Reaction of the boronic acids or esters can be carried out with starting material 11 in the presence of an appropriate palladium catalyst, such as palladium tetrakistriphenylphosphine, and a base such as sodium carbonate and the like, to provide pyrazolopyrimidines 12.

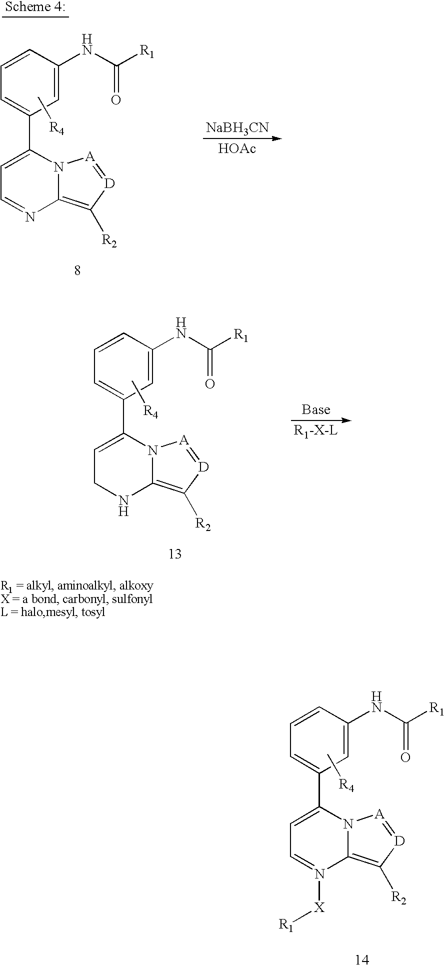

As described in Scheme 4, dihydropyrazolopyrimidine compounds, 14, can be prepared from the corresponding pyrazolopyrimidines 8 by a partial reduction of the pyrimidine ring, by using for example a reducing agent such as sodium cyanoborohydride, according to a procedure described in: EP 264773 (1988) in the presence of a weak acid such as acetic acid to give the resulting intermediate dihydro[1,5-1]pyrazolopyrimidine 13, which can be subsequently reacted with a variety of electrophiles such as, but not restricted to acyl halides, alkyl, allyl and propargyl halides, anhydrides, sulfonyl halides and alkyl sulfonates together with a base, such as a trialkylamine, sodium hydride, potassium carbonate and the like to provide compounds of formula 14.

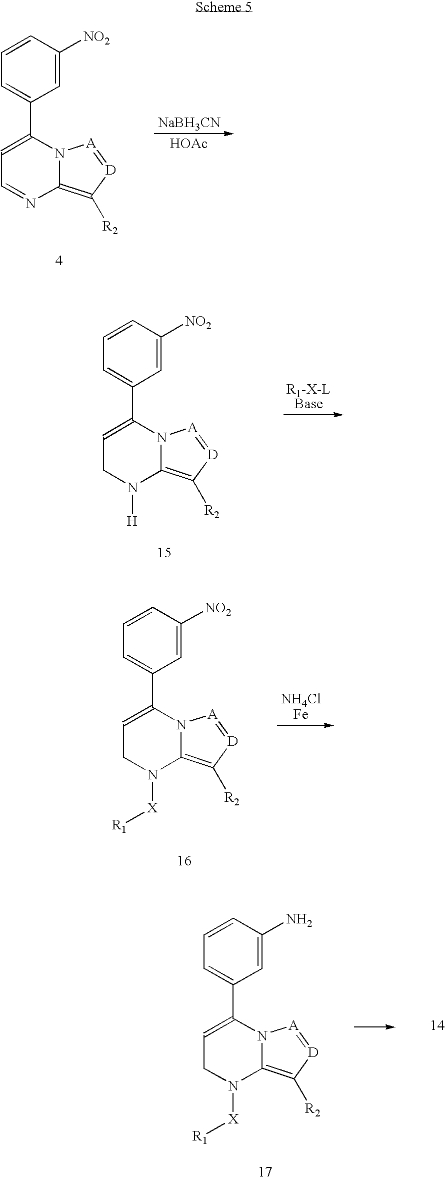

Compounds of formula 14 may also be prepared according to the route shown in Scheme 5. Thus, nitroaryl pyrazolopyrimidine 4 can be reduced with sodium cyanoborohydride in acetic acid, or an equivalent reducing agent, to give dihydropyrazolopyrimidine 15. Functionalization of the pyrimidine nitrogen by reaction with electrophiles as described in Scheme 4 provides intermediates 16. The reduction of nitro group of compound 16 with reducing agents such as Fe, SnC1₂-xH₂O, catalytic hydrogenation and the like, gives the aniline 17. Aniline 17 can then be reacted with acylating agents such as an acyl chloride or carboxylic acid anhydride, in the presence of a base such as pyridine, triethylamine, N-methylmorpholine, 4-dimethylaminopyridine and the like, to give amides 14. Alternatively, amides 14 can be prepared from the reaction of aniline 17 with a carbamate intermediate generated in situ by treating a carboxylic acid with an alkyl chloroformate in the presence of a base as defined above. Amides 14 can also be prepared by reacting with a carboxylic acid 7 in the presence of coupling reagents like 1-(3-Dimethylaminopropyl)-3-ethylcarbodiimide hydrochloride(EDCI), 1,3-dicyclohexylcarbodiimide (DCC), 1-hydroxybenzotriazole hydrate (HOBT), O-(benzotriazol-1-yl)-N,N,N′,N′-tetramethyluronium hexafluorophosphate(HBTU) and the like, in the presence of a base as defined above.

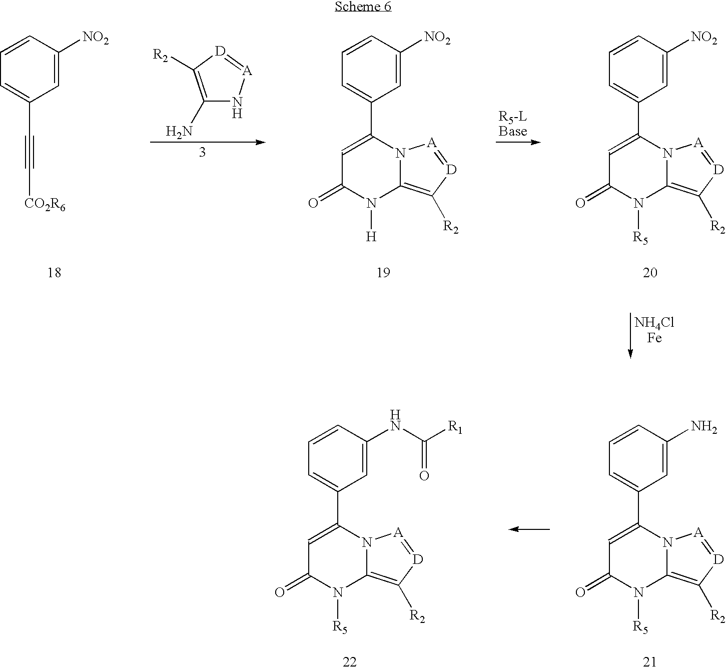

Compounds of Formula I of the invention wherein Q1 and Q2 together form a carbonyl group may be prepared as shown in Scheme 6. Reaction of acetylenic ester 18 with the appropriately substituted amine 3 either by heating the two components together neat, or by heating them in a solvent such as ethanol, THF, dioxane or acetic acid, provides compounds of structure 19. A base such as a sodium alkoxide, sodium hydride, or potassium carbonate may be used in this transformation. Derivatization of the pyrimidinone NH by reaction with alkyl halides provides 20 that can then be reduced to the desired aniline intermediate 21 using reducing agents such as Fe, SnC1₂-xH₂O, catalytic hydrogenation and the like. Acylation of aniline intermediate 21 with acylating agents such as an acyl chloride or carboxylic acid anhydride, in the presence of a base such as pyridine, triethylamine, N-methylmorpholine, 4-dimethylaminopyridine and the like, then gives amides 22. Alternatively, amides 22 can be prepared from the reaction of aniline with carbamate intermediate generated in situ by treating a carboxylic acid with an alkyl chloroformate in the presence of base as defined above. Amides 22 can also be prepared by reacting with a carboxylic acid in the presence of coupling reagents like EDCI, DCC, HOBT, HBTU and the like, in the presence of base as defined above.

The compounds of Formula (I) may be obtained as inorganic or organic salts using methods known to those skilled in the art (Richard C. Larock, Comprehensive Organic Transformations, VCH publishers, 411-415, 1989). It is well known to one skilled in the art that an appropriate salt form is chosen based on physical and chemical stability, flowability, hydroscopicity and solubility.

Pharmaceutically acceptable salts of the compounds of Formula (I) with an acidic moiety may be formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be formed from organic and inorganic acids. For example salts may be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids. The compounds can also be used in the form of esters, carbamates and other conventional prodrug forms, which when administered in such form, convert to the active moiety in vivo.

The present invention accordingly provides a pharmaceutical composition which comprises a compound of this invention in combination or association with a pharmaceutically acceptable carrier. In particular, the present invention provides a pharmaceutical composition which comprises an effective amount of a compound of this invention and a pharmaceutically acceptable carrier.

### Standard Pharmacological Test Procedures

Evaluation of representative compounds of this invention in standard pharmacological test procedures indicated that the compounds of this invention possess significant anticancer activity and are inhibitors of protein tyrosine kinases and in particular B-raf. Based on the activity shown in the standard pharmacological test procedures, the compounds of this invention are therefore useful as antineoplastic agents. In particular, these compounds are useful in treating, inhibiting the growth of, or eradicating neoplasms such as those of the breast, kidney, bladder, mouth, larynx, esophagus, stomach, colon, ovary, lung, pancreas, liver, prostate and skin.

In addition to having antineoplastic properties, the compounds of the present invention are expected to be useful in treating a variety of protein tyrosine kinase associated disorders including, but not limited to, osteoporosis, osteoarthritis, restenosis, atherosclerosis, fibroplasia, angiofibromas, hemangiomas, diabetes, acute and chronic nephropathies, Kaposi's sarcoma, atheroma, neovascular glaucoma, neovascularization associated with macular degeneration, rheumatoid arthritis, psoriatic arthritis, transplant rejection, T-cell mediated hypersensitivity diseases, including gluten-sensitive enteropathy (Celiac disease), contact and delayed-type hypersensitivity, psoriasis, contact dermatitis, protection from ischemic or reperfusion injury such as that incurred during organ transplantation, stroke or myocardial infarction, transplantation tolerance induction, lupus, graft versus host disease, glomerulonephritis, serum sickness, respiratory and skin allergies, autoimmune alopecia, pernicious anemia, Hashimoto's thyroiditis, autoimmune hyperthyroidism, Addison's disease, multiple sclerosis, inflammatory bowel disease, acute inflammatory responses (for example acute respiratory distress syndrome), Behcet's disease, atopic dermatitis, systemic sclerosis and eczema.

### Biological Test Procedure(s)

TITLE: B-Raf Kinase to MEK1 ELISA
PURPOSE: To discover B-Raf Kinase inhibitors that can inhibit growth of tumor cells which contain oncogenic forms of Receptor Tyrosine Kinases or K-Ras, or B-Raf kinase.

### Materials and Methods:

Reagents: Flag/GST-tagged recombinant human B-Raf produced in Sf9 insect cells, human non-active Mek-1-GST (recombinant protein produced in E. coli); and a phospho-MEK1 specific poly-clonal Ab from Cell Signaling Technology (cat. #9121).

### B-Raf1 Kinase Assay Procedure:

B-Raf-1 is used to phosphorylate GST-MEK1. MEK1 phosphorylation is measured by a phospho-specific antibody (from Cell Signaling Technology, cat. #9121) that detects phosphorylation of two serine residues at positions 217 and 221 on MEK1.

### Kinase Assay Protocol

### B-Raf Assay Stock Solutions:

1. Assay Dilution Buffer (ADB): 20 mM MOPS, pH 7.2, 25 mM β-glycerol phosphate, 5 mM EGTA, 1 mM sodium orthovanadate, 1 mM dithiothreitol.

2. Magnesium/ATP Cocktail: 200 μM cold ATP and 40 mM magnesium chloride in ADB.

4. Active Kinase: Active B-Raf: used at ˜20 ng per assay point.

5. Non-active GST-MEK1: Use at 100 nM (50 nM final).

6. TBST—Tris (50 mM, pH 7.5), NaCl (150 mM), Tween-20 (0.05%)

7. Anti-GST Ab (Pharmacia)

8. Anti-rabbit Ab/Europium conjugate (Wallac)

### Assay Procedure:

1. Add 25 μl of ADB containing B-Raf and Mek per assay (i.e. per well of a 96 well plate)

2. Add 25 μl of 0.2 mM ATP and 40 mM magnesium chloride in ADB.

3. Incubate for 60 minutes at RT in a shaking incubator.

4. Transfer this mixture to an anti-GST Ab coated 96 well plate (Nunc Immunosorb plates coated o/n with a-GST.

5. Incubate for 60 minutes at 30° C. in a shaking incubator

6. Wash 3× with TBST, add Anti-Phospho MEK1 (1:1000)

7. Incubate for 60 minutes at 30° C. in a shaking incubator

8. Wash 3× with TBST, add Anti-rabbit Ab/Europium conjugate (Wallac) (1:500)

9. Incubate for 60 minutes at 30° C. in a shaking incubator

10. Add 100 ul of Wallac Delfia Enhancement Solution and shake for 10 minutes.

11. Read plates in Wallac Victor model Plate Reader.

12. Collect data analyze in Excel for single point and IC50 determinations.

### Analysis of Results:

Single point assay—% inhibition at 10 mg/ml (% Inhibition=1−cpd.treated sample/untreated control)
IC50 determinations—done on cpds from single pt assays with >80% inhibition. Typically Raf-1 assay is run at cpd concentrations from 10 μM to 30 nM in half log dilutions. (% inhibition is determined for each cpd concentration)
Data handling—Data is analyzed in Excel, and will transferred to Radis.

### References:

1.) Davies H, et al. (2002) Nature 417:906

2.) Rajagopalan H, et al (2002) Nature 418:934

3.) Mallon R, et al (2001) Anal. Biochem. 294:48

| Example# | Name | B-Raf IC50(μM) |
|---|---|---|
| Example 1 | N-[3-(4,5-dihydropyrazolo[1,5-a]pyrimidin-7- | >10 |
| | yl)phenyl]-3-(trifluoromethyl)benzamide | |
| Example 2 | N-{3-[4-(methylsulfonyl)-4,5-dihydropyrazolo[1,5- | 0.6832 |
| | a]pyrimidin-7-yl]phenyl}-3- | |
| | (trifluoromethyl)benzamide | |
| Example 3 | methyl 7-(3-{[3- | 0.1634 |
| | (trifluoromethyl)benzoyl]amino}phenyl)pyrazolo[1,5- | |
| | a]pyrimidine-4(5H)-carboxylate | |
| Example 4 | ethyl 7-(3-{[3- | 0.3702 |
| | (trifluoromethyl)benzoyl]amino}phenyl)pyrazolo[1,5- | |
| | a]pyrimidine-4(5H)-carboxylate | |
| Example 5 | N-isopropyl-7-(3-{[3- | 1.6184 |
| | (trifluoromethyl)benzoyl]amino}phenyl)pyrazolo[1,5- | |
| | a]pyrimidine-4(5H)-carboxamide | |
| Example 6 | tert-butyl 7-(3-{[3- | 0.8153 |
| | (trifluoromethyl)benzoyl]amino}phenyl)pyrazolo[1,5- | |
| | a]pyrimidine-4(5H)-carboxylate | |
| Example 7 | N-[3-(4-but-2-yn-1-yl-4,5-dihydropyrazolo[1,5- | 0.8677 |
| | a]pyrimidin-7-yl)phenyl]-3- | |
| | (trifluoromethyl)benzamide | |
| Example 8 | ethyl 7-(3-{[3- | 0.2777 |
| | (trifluoromethyl)benzoyl]amino}phenyl)-4,5- | |
| | dihydropyrazolo[1,5-a]pyrimidine-3-carboxylate | |
| Example 9 | N-ethyl-7-(3-{[3- | >10 |
| | (trifluoromethyl)benzoyl]amino}phenyl)pyrazolo[1,5- | |
| | a]pyrimidine-4(5H)-carboxamide | |
| Example 10 | N-[3-(4-acetyl-4,5-dihydropyrazolo[1,5-a]pyrimidin- | 3.77 |
| | 7-yl)phenyl]-3-(trifluoromethyl)benzamide | |
| Example 11 | ethyl 4-acetyl-7-(3-{[3- | 0.572 |
| | (trifluoromethyl)benzoyl]amino}phenyl)-4,5- | |
| | dihydropyrazolo[1,5-a]pyrimidine-3-carboxylate | |
| Example 12 | tert-butyl 7-(3-{(tert-butoxycarbonyl)[3- | >10 |
| | (trifluoromethyl)benzoyl]amino}phenyl)pyrazolo[1,5- | |
| | a]pyrimidine-4(5H)-carboxylate | |
| Example 13 | 3-ethyl 4-methyl 7-(3-{[3- | 0.126 |
| | (trifluoromethyl)benzoyl]amino}phenyl)pyrazolo[1,5- | |
| | a]pyrimidine-3,4(5H)-dicarboxylate | |
| Example 14 | tert-butyl {2-oxo-2-[7-(3-{[3- | >10 |
| | (trifluoromethyl)benzoyl]amino}phenyl)pyrazolo[1,5- | |
| | a]pyrimidin-4(5H)-yl]ethyl}carbamate | |
| Example 15 | N-[3-(4-glycyl-4,5-dihydropyrazolo[1,5-a]pyrimidin- | >10 |
| | 7-yl)phenyl]-3-(trifluoromethyl)benzamide | |
| Example 16 | N-{3-[4-(N-acetylglycyl)-4,5-dihydropyrazolo[1,5- | 1.77 |
| | a]pyrimidin-7-yl]phenyl}-3- | |
| | (trifluoromethyl)benzamide | |
| Example 17 | tert-butyl {3-oxo-3-[7-(3-{[3- | 0.385 |
| | (trifluoromethyl)benzoyl]amino}phenyl)pyrazolo[1,5- | |
| | a]pyrimidin-4(5H)-yl]propyl}carbamate | |
| Example 18 | N-[3-(4-β-alanyl-4,5-dihydropyrazolo[1,5- | >10 |
| | a]pyrimidin-7-yl)phenyl]-3- | |
| | (trifluoromethyl)benzamide | |
| Example 19 | N-{3-[4-(N-acetyl-β-alanyl)-4,5-dihydropyrazolo[1,5- | 0.716 |
| | a]pyrimidin-7-yl]phenyl}-3- | |
| | (trifluoromethyl)benzamide | |
| Example 20 | N-[4-chloro-3-(4,5-dihydropyrazolo[1,5-a]pyrimidin- | 0.161 |
| | 7-yl)phenyl]-3-(trifluoromethyl)benzamide | |

Based on the results obtained for representative compounds of this invention, the compounds of this invention are antineoplatic agents which are useful in treating, inhibiting the growth of, or eradicating neoplasms. In particular, the compounds of this invention are useful in treating, inhibiting the growth of, or eradicating neoplasms that either express Src or raf or neoplasms that depend at least in part on the Src or raf pathways. Such neoplasms include those of the breast, kidney, bladder, mouth, larynx, esophagus, stomach, colon, ovary, lung, pancreas, skin, liver, prostate or brain. Based on the results obtained, the compounds of this invention are also useful in the treatment of osteoporosis.

As may be appreciated from the data, the compounds according to the invention are endowed with valuable biological properties useful in the treatment of certain diseases that are the result of deregulation of protein kinases.

The compounds of this invention may be formulated neat or may be combined with one or more pharmaceutically acceptable carriers for administration. For example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to about 1000 mg/kg of animal body weight, optionally given in divided doses two to four times a day, or in sustained release form. For most large mammals the total daily dosage is from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The compounds of this invention may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compounds is preferred.

In some cases it may be desirable to administer the compounds directly to the airways in the form of an aerosol.

The compounds of this invention may also be administered parenterally or intraperitoneally. Solutions or suspensions of these active compounds as a free base or pharmacologically acceptable salt may be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (e.g., glycerol, propylene glycol and liquid polyethylene glycol), suitable mixtures thereof, and vegetable oils.

For the treatment of cancer, the compounds of this invention may be administered in combination with other antitumor substances or with radiation therapy. These other substances or radiation treatments may be given at the same or at different times as the compounds of this invention. These combined therapies may effect synergy and result in improved efficacy. For example, the compounds of this invention may be used in combination with mitotic inhibitors such as taxol or vinblastine, alkylating agents such as cisplatin or cyclophosamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, and antiestrogens such as tamoxifen.

As used in accordance with this invention, the term providing an effective amount of a compound means either directly administering such compound, or administering a prodrug, derivative, or analog which will form an effective amount of the compound within the body.

The invention will be more fully described in conjunction with the following specific examples which are not to be construed as limiting the scope of the invention.

### EXAMPLE 1

### N-[3-(4,5-dihydropyrazolo[1,5-a]pyrimidin-7-yl)phenyl]-3-(trifluoromethyl)benzamide

Step 1: N-(3-Acetyl-phenyl)-3-trifluoromethyl-benzamide: To a solution of 3-amino acetophenone (3 g, 22 mmol) in pyridine (18 mL) is added 3-trifluoromethyl benzoyl chloride (5 g, 24 mmol) and heated at 50° C. overnight. The solution is then concentrated and the crude product is taken to next step without further purification.

Step2: N-[3-(3-Dimethylamino-acryloyl)-phenyl]-3-trifluoromethyl-benzamide: The above benzamide is taken up in N,N dimethylformamide dimethyl acetal (5 mL) and heated for 7 hours at 80° C. Resulting solution is then concentrated and used in the next step without any further purification.

Step 3: N-(3-pyrazolo[1,5-a]pyrimidin-7-ylphenyl)-3-(trifluoromethyl)benzamide: To a solution of 4.40 g (20 mmol) of N-[3-(3-dimethylamino-acryloyl)-phenyl]-3-trifluoromethyl-benzamide in 20 mL of acetic acid is added 3-aminopyrazole (1.99 g, 24 mmol) and the resulting mixture is heated at 110° C. for 4 hours. The reaction mixture was then cooled and the precipitate is collected by filtration. The solid is washed with hexanes and water and then dried in vacuo to provide 4.38 g (91%) of N-(3-pyrazolo[1,5-a]pyrimidin-7-ylphenyl)-3-(trifluoromethyl)benzamide.

Step 4: To a suspension of 3.82 g (10 mmol) of N-(3-pyrazolo[1,5-a]pyrimidin-7-ylphenyl)-3-(trifluoromethyl)benzamide in 50 ml of acetic acid is added in portions 1.38 g (22 mmol) of sodium cyanoborohydride at room temperature under nitrogen (following the procedure described in: EP 264773(1988)). The mixture is stirred at room temperature for 2 hours. After removal of the volatile material, 400 ml of ethyl acetate is added. The organic phase is neutralized with a saturated sodium bicarbonate solution and washed with a saturated sodium chloride solution and dried over magnesium sulfate. Following the removal of solvents in vacuo, 2.63 g (68% yield) of N-[3-(4,5-dihydropyrazolo[1,5-a]pyrimidin-7-yl)phenyl]-3-(trifluoromethyl)benzamide is obtained.

MS: 385.2 (M+H)⁺

### EXAMPLE 2

### N-{3-[4-(Methylsulfonyl)-4,5-dihydropyrazolo[1,5-a]pyrimidin-7-yl]phenyl}-3-(trifluoromethyl)benzamide

To the stirred solution of 307 mg (0.8 mmol) of N-[3-(4,5-dihydropyrazolo[1,5-a]pyrimidin-7-yl)phenyl]-3-(trifluoromethyl)benzamide and 0.28 ml of diisopropylethylamine (Hunig's base) in 5 ml of DMF is added 92 mg (1.2 mmol) of methanesulfonyl chloride. The solution is stirred at room temperature overnight. After removal of the volatile materials, 80 ml of ethyl acetate is added. The organic phase is washed with water and a saturated sodium chloride solution, dried over magnesium sulfate. Following the removal of solvents in vacuo, 181 mg (49% yield) of N-{3-[4-(methylsulfonyl)-4,5-dihydropyrazolo[1,5-a]pyrimidin-7-yl]phenyl}-3-(trifluoromethyl)benzamide is obtained by flash column chromatography, eluting with ethyl acetate/hexane.

MS: 461.3 (M−H)⁻

### EXAMPLE 3

### Methyl 7-(3-{[3-(trifluoromethyl)benzoyl]amino}phenyl)pyrazolo[1,5-a]pyrimidine-4(5H)-carboxylate

To 307 mg (0.8 mmol) of N-[3-(4,5-dihydropyrazolo[1,5-a]pyrimidin-7-yl)phenyl]-3-(trifluoromethyl)benzamide and 113 mg (1.2 mmol) of methyl chloroformate in 5 mL of DMF is added 0.28 ml of Hunig's base. The solution is stirred at 50° C. overnight. After removal of the volatile material, 80 ml of ethyl acetate is added. The organic phase is washed with water and a saturated sodium chloride solution, dried over magnesium sulfate. Following the removal of solvents in vacuo, 57 mg (16% yield) of methyl 7-(3-{[3-(trifluoromethyl)benzoyl]amino}phenyl)pyrazolo[1,5-a]pyrimidine-4(5H)-carboxylate is obtained after flash column chromatography eluting with ethyl acetate/hexane.

MS: 443.3 (M+H)⁺

### EXAMPLE 4

### Ethyl 7-(3-{[3-(trifluoromethyl)benzoyl]amino}phenyl)pyrazolo[1,5-a]pyrimidine-4(5H)-carboxylate

According to the procedure of Example 3, the reaction of 307 mg of N-[3-(4,5-dihydropyrazolo[1,5-a]pyrimidin-7-yl)phenyl]-3-(trifluoromethyl)benzamide with 130 mg of ethyl chloroformate provided 53 mg (15% yield) of ethyl 7-(3-{[3-(trifluoromethyl)benzoyl]amino}phenyl)pyrazolo[1,5-a]pyrimidine-4(5H)-carboxylate.

MS: 457.3 (M+H)⁺

### EXAMPLE 5

### N-isopropyl-7-(3-{[3-(trifluoromethyl)benzoyl]amino}phenyl)pyrazolo[1,5-a]pyrimidine-4(5H)-carboxamide

To a stirred solution of 307 mg (0.8 mmol) of N-[3-(4,5-dihydropyrazolo[1,5-a]pyrimidin-7-yl)phenyl]-3-(trifluoromethyl)benzamide in 5 ml of DMF is added 70 mg sodium hydride (60%) at 0° C., followed by 82 mg (0.9 mmol) of isopropyl isocyanate. The solution is stirred at room temperature overnight. After removal of the volatile material, 80 ml of ethyl acetate is added. The organic phase is washed with water and a saturated sodium chloride solution, dried over magnesium sulfate. Following the removal of solvents in vacuo, 42 mg (11% yield) of N-isopropyl-7-(3-{[3-(trifluoromethyl)benzoyl]amino}phenyl)pyrazolo[1,5-a]pyrimidine-4(5H)-carboxamide is obtained by flash column chromatography, eluting with ethyl acetate/hexane.

MS: 470.4 (M+H)⁺

### EXAMPLE 6

### tert-Butyl 7-(3-{[3-(trifluoromethyl)benzoyl]amino}phenyl)pyrazolo[1,5-a]pyrimidine-4(5H)-carboxylate

To a stirred solution of 307 mg (0.8 mmol) of N-[3-(4,5-dihydropyrazolo[1,5-a]pyrimidin-7-yl)phenyl]-3-(trifluoromethyl)benzamide in 5 ml of DMF is added 70 mg sodium hydride (60%) at 0° C., followed by 349 mg (1.6 mmol) of di-tert-butyl dicarbonate. The solution is stirred at room temperature overnight. After removal of the volatile material, 80 ml of ethyl acetate is added. The organic phase is washed with water and a saturated sodium chloride solution, dried over magnesium sulfate. Following the removal of solvents in vacuo, 38 mg (7% yield) of tert-butyl 7-(3-{[3-(trifluoromethyl)benzoyl]amino}phenyl)pyrazolo[1,5-a]pyrimidine-4(5H)-carboxylate is obtained by flash column chromatography, eluting with ethyl acetate/hexane.

MS: 485.1 (M+H)⁺

### EXAMPLE 7

### N-[3-(4-But-2-yn-1-yl-4,5-dihydropyrazolo[1,5-a]pyrimidin-7-yl)phenyl]-3-(trifluoromethyl)benzamide

To a stirred solution of 307 mg (0.8 mmol) of N-[3-(4,5-dihydropyrazolo[1,5-a]pyrimidin-7-yl)phenyl]-3-(trifluoromethyl)benzamide and 0.28 ml of Hunig's base in 5 ml of DMF is added 128 mg (0.9 mmol) of 1-bromo-2-butyne. The solution is stirred at 50° C. overnight. After removal of the volatile material, 80 ml of ethyl acetate is added. The organic phase is washed with water and a saturated sodium chloride solution, dried over magnesium sulfate. Following the removal of solvents in vacuo, 99 mg (28% yield) of N-[3-(4-but-2-yn-1-yl-4,5-dihydropyrazolo[1,5-a]pyrimidin-7-yl)phenyl]-3-(trifluoromethyl)benzamide is obtained by flash column chromatography eluting with ethyl acetate/hexane.

MS: 437.3 (M+H)⁺

### EXAMPLE 8

### Ethyl 7-(3-{[3-(trifluoromethyl)benzoyl]amino}phenyl)-4,5-dihydropyrazolo[1,5-a]pyrimidine-3-carboxylate

Step 1: 3-(Dimethylamino)-1-(3-nitrophenyl)-2-propen-1-one 3-Nitroacetophenone (5.0 g, 30.3 mmol) in dimethylformamide-dimethylacetal (10 mL) is heated at reflux overnight. The reaction mixture is cooled to room temperature and evaporated to remove the volatiles. The residue is slurried in ethyl ether and the suspension is filtered and washed with ether to give 10.5 g (79%) of 3-(dimethylamino)-1-(3-nitrophenyl)-2-propen-1-one, 104-105° C.

Step 2: 7-(3-Nitro-phenyl)-pyrazolo[1,5-a]pyrimidine-3-carboxylic acid ethyl ester to a solution of 3-dimethylamino-1-(3-nitro-phenyl)-propenone (3 mmol) in acetic acid is added 3 amino-4 carbethoxyprazole (3.1 mmol) and heated at 80° C. overnight. The solution is concentrated and the tan solid obtained is taken to the next step without further purification.

Step 3: 7-(3-Amino-phenyl)-pyrazolo[1,5-a]pyrimidine-3-carboxylic acid ethyl ester: A 2.0 L three neck flask equipped with mechanical stirrer is added 7-(3-nitro-phenyl)-pyrazolo[1,5-a]pyrimidine-3-carboxylic acid ethyl ester (86 mmol), and ammonium chloride (428 mmol) in methanol (200 mL) and water (200 mL). The mixture is stirred for 5 minutes. Iron powder (343 mmol) is added slowly with stirring followed by an additional 200 mL of methanol and 200 mL of water. The reaction mixture is heated gradually to reflux and maintained at reflux overnight, cooled to room temperature and filtered. The red solid cake is washed thoroughly with hot methanol and hot ethyl acetate. The combined filtrates are evaporated to give 7-(3-Amino-phenyl)-pyrazolo[1,5-a]pyrimidine-3-carboxylic acid ethyl ester as a light brown solid. The crude product is used directly for the next step without further purification.

Step 4: Pyrazolo[1,5-a]pyrimidine-3-carboxylic acid,7-[3-[[3-(trifluoromethyl)-benzoyl]amino]phenyl]-,ethyl ester to a solution of 7-(3-amino-phenyl)-pyrazolo[1,5-a]pyrimidine-3-carboxylic acid ethyl ester (0.15 mmol) in pyridine is added 3-(trifluoromethyl) benzoyl chloride (0.17 mmol) and stirred at room temperature for 18 h. The reaction mixture is concentrated and purified by HPLC.

Step 5: To a suspension of 1.36 g (3 mmol) of ethyl 7-(3-{[3-(trifluoromethyl)benzoyl]amino}phenyl)pyrazolo[1,5-a]pyrimidine-3-carboxylate, in 20 ml of acetic acid is added 0.41 g (6.6 mmol) of sodium cyanoborohydride in portions at room temperature under nitrogen (following the procedure described in: EP 264773(1988)). The mixture is stirred at room temperature for 2 hr. The solid precipitate is filtered, washed with a saturated sodium bicarbonate solution, water and dried over magnesium sulfate. Following the removal of solvents in vacuo, 1.20 g (87% yield) of ethyl 7-(3-{[3-(trifluoromethyl)benzoyl]amino}phenyl)-4,5-dihydropyrazolo[1,5-a]pyrimidine-3-carboxylate is obtained.

MS: 457.3 (M+H)⁺

### EXAMPLE 9

### N-Ethyl-7-(3-{[3-(trifluoromethyl)benzoyl]amino}phenyl)pyrazolo[1,5-a]pyrimidine-4(5H)-carboxamide

To a stirred solution of 307 mg (0.8 mmol) of N-[3-(4,5-dihydropyrazolo[1,5-a]pyrimidin-7-yl)phenyl]-3-(trifluoromethyl)benzamide in 5 ml of DMF is added 70 mg sodium hydride (60%) at 0° C., followed by 85 mg (1.2 mmol) of ethyl isocyanate. The solution is stirred at room temperature overnight. After removal of the volatile material, 80 ml of ethyl acetate is added. The organic phase is washed with water and a saturated sodium chloride solution, then dried over magnesium sulfate. Following the removal of solvents in vacuo, 28 mg (8% yield) of N-ethyl-7-(3-{[3-(trifluoromethyl)benzoyl]amino}phenyl)pyrazolo[1,5-a]pyrimidine-4(5H)-carboxamide is obtained by flash column chromatography, eluting with ethyl acetate/hexane.

MS: 456.0 (M+H)⁺

### EXAMPLE 10

### N-[3-(4-Acetyl-4,5-dihydropyrazolo[1,5-a]pyrimidin-7-yl)phenyl]-3-(trifluoromethyl)benzamide

To a stirred solution of 307 mg (0.8 mmol) of N-[3-(4,5-dihydropyrazolo[1,5-a]pyrimidin-7-yl)phenyl]-3-(trifluoromethyl)benzamide in 5 ml of DMF is added 70 mg sodium hydride (60%) at 0° C., followed by 98 mg (1.2 mmol) of acetic anhydride. The solution is stirred at room temperature overnight. After removal of the volatile material, 80 ml of ethyl acetate is added. The organic phase is washed with water and a saturated sodium chloride solution, dried over magnesium sulfate. Following the removal of solvents in vacuo, 94 mg (27% yield) of N-[3-(4-acetyl-4,5-dihydropyrazolo[1,5-a]pyrimidin-7-yl)phenyl]-3-(trifluoromethyl)benzamide is obtained by flash column chromatography, eluting with ethyl acetate/hexane.

MS: 427.0 (M+H)⁺

### EXAMPLE 11

### Ethyl 4-acetyl-7-(3-{[3-(trifluoromethyl)benzoyl]amino}phenyl)-4,5-dihydropyrazolo[1,5-a]pyrimidine-3-carboxylate

To a stirred solution of 365 mg (0.8 mmol) of ethyl 7-(3-{[3-(trifluoromethyl)benzoyl]amino}phenyl)-4,5-dihydropyrazolo[1,5-a]pyrimidine-3-carboxylate, the product of Example 8, in 5 ml of DMF is added 70 mg sodium hydride (60%) at 0° C., followed by 122 mg (1.2 mmol) of acetic anhydride. The solution is stirred at room temperature overnight. After removal of the volatile material, 80 ml of ethyl acetate is added. The organic phase is washed with water and a saturated sodium chloride solution, dried over magnesium sulfate. Following the removal of solvents in vacuo, 152 mg (38% yield) of ethyl 4-acetyl-7-(3-{[3-(trifluoromethyl)benzoyl]amino}phenyl)-4,5-dihydropyrazolo[1,5-a]pyrimidine-3-carboxylate is obtained by flash column chromatography, eluting with ethyl acetate/hexane.

MS: 499.3 (M+H)⁺

### EXAMPLE 12

### tert-Butyl 7-(3-{(tert-butoxycarbonyl)[3-(trifluoromethyl)benzoyl]amino}phenyl)pyrazolo[1,5-a]pyrimidine-4(5H)-carboxylate 4(5H)-carboxylate

To a stirred solution of 307 mg (0.8 mmol) of N-[3-(4,5-dihydropyrazolo[1,5-a]pyrimidin-7-yl)phenyl]-3-(trifluoromethyl)benzamide, the product of Example 1, in 5 ml of DMF at 0° C. was added 70 mg of 60% sodium hydride, followed by 349 mg (1.6 mmol) of di-tert-butyl dicarbonate. The resulting mixture was stirred at room temperature overnight. After removal of the volatile material in vacuo, 80 ml of ethyl acetate was added. The organic phase was washed with water and saturated sodium chloride solution, dried over magnesium sulfate and filtered. The filtrate was concentrated in vacuo and the residue was purified by flash column chromatography on silica gel eluting with ethyl acetate/hexane with to give 207 mg (44% yield) of tert-Butyl 7-(3-{(tert-butoxycarbonyl)[3-trifluoromethyl)benzoyl]amino}phenyl)pyrazolo[1,5-a]pyrimidine-4(5H)-carboxylate 4(5H)-carboxylate. MS (ESI) m/z: 585.1 (M+H)⁺

### EXAMPLE 13

### 3-Ethyl 4-methyl 7-(3-{[3-(trifluoromethyl)benzoyl]amino}phenyl)pyrazolo[1,5-a]pyrimidine-3,4(5H)-dicarboxylate

To a stirred solution of 365 mg (0.8 mmol) of ethyl 7-(3-{[3-(trifluoromethyl)benzoyl]amino}phenyl)-4,5-dihydropyrazolo[1,5-a]pyrimidine-3-carboxylate, the product of Example 8, in 5 ml of DMF at 0° C. was added 70 mg of 60% sodium hydride, followed by 113 mg (1.2 mmol) of methyl chloroformate. The solution was stirred at room temperature overnight. After removal of the volatile material in vacuo, 80 ml of ethyl acetate was added. The organic phase was washed with water and saturated sodium chloride solution, dried over magnesium sulfate, filtered and concentrated in vacuo. The residue was purified by flash column chromatography on silica gel eluting with ethyl acetate/hexane to provide 142 mg (34% yield) of 3-ethyl 4-methyl 7-(3-{[3-(trifluoromethyl)benzoyl]amino}phenyl)pyrazolo[1,5-a]pyrimidine-3,4(5H)-dicarboxylate. MS (ESI) m/z: 515.3 (M+H)⁺

### EXAMPLE 14

### tert-Butyl {2-oxo-2-[7-(3-{[3-(trifluoromethyl)benzoyl]amino}phenyl)pyrazolo[1,5-a]pyrimidin-4(5H)-yl]ethyl}carbamate

Step 1: 7-(3-Nitrophenyl)-4,5-dihydropyrazolo[1,5-a]pyrimidine to a suspension of 2.40 g (10 mmol) of 7-(3-nitrophenyl)pyrazolo[1,5-a]pyrimidine in 50 ml of acetic acid was added in portions 1.38 g (22 mmol) of sodium cyanoborohydride at room temperature under N₂. The mixture was stirred at room temperature for 2 hr. After removal of the volatile material in vacuo, 400 ml of ethyl acetate was added. The organic phase was neutralized with saturated sodium bicarbonate solution, washed with brine, dried over magnesium sulfate, filtered and concentrated in vacuo to provide 2.01 g (83% yield) of 7-(3-nitrophenyl)-4,5-dihydropyrazolo[1,5-a]pyrimidine. MS (ESI) m/z: 243.2 (M+H)⁺

Step 2: tert-Butyl {2-[7-(3-nitrophenyl)pyrazolo[1,5-a]pyrimidin-4(5H)-yl]-2-oxoethyl}carbamate to a stirred solution of 578 mg (3.3 mmol) of N-tert-butoxycarbonylglycine in 5 ml of DMF at 0° C. was added 1.72 g (3.9 mmol) of BOP, followed by 0.7 ml of Hunig's base, and 726 mg (1 mmol) of 7-(3-nitrophenyl)-4,5-dihydropyrazolo[1,5-a]pyrimidine. The solution was stirred at 45° C. overnight. After removal of the volatile material in vacuo, 200 ml of ethyl acetate was added. The organic phase was washed with water and saturated sodium chloride solution, dried over magnesium sulfate, filtered and concentrated in vacuo. The residue was obtained by flash column chromatography on silica gel eluting with ethyl acetate/hexane to give 480 mg (40% yield) of tert-butyl {2-[7-(3-nitrophenyl)pyrazolo[1,5-a]pyrimidin-4(5H)-yl]-2-oxoethyl}carbamate. MS (ESI) m/z: 400.3 (M+H)⁺

Step 3: tert-Butyl {2-[7-(3-aminophenyl)pyrazolo[1,5-a]pyrimidin-4(5H)-yl]-2-oxoethyl}carbamate to a stirred solution of 240 mg (0.6 mmol) of tert-butyl {2-[7-(3-nitrophenyl)pyrazolo[1,5-a]pyrimidin-4(5H)-yl]-2-oxoethyl}carbamate in 8 ml of ethanol and 3 ml of water was added 289 mg (5.4 mmol) of ammonium chloride and 101 mg (1.8 mmol) of iron powder. The reaction mixture was then refluxed for 3 hr. After filtration, the solution was concentrated in vacuo and 150 ml of ethyl acetate was added. The organic phase was washed with water and saturated sodium chloride solution, dried over magnesium sulfate, filtered and concentrated in vacuo to provide 209 mg (94% yield) of tert-butyl {2-[7-(3-aminophenyl)pyrazolo[1,5-a]pyrimidin-4(5H)-yl]-2-oxoethyl}carbamate. This material was pure enough without further purification. MS (ESI) m/z: 370.3 (M+H)⁺

Step 4: tert-Butyl {2-oxo-2-[7-(3-{[3-(trifluoromethyl)benzoyl]amino}phenyl)pyrazolo[1,5-a]pyrimidin-4(5H)-yl]ethyl}carbamate to a stirred solution of 176 mg (0.48 mmol) of tert-butyl {2-[7-(3-aminophenyl)pyrazolo[1,5-a]pyrimidin-4(5H)-yl]-2-oxoethyl}carbamate and 57 mg of pyridine in 5 ml of dichloromethane was added 104 mg (0.5 mmol) of 3-(trifluoromethyl)benzoyl chloride. The solution was stirred at room temperature overnight and then 120 ml of dichloromethane was added. The organic phase was washed with 0.1 N HCl solution, water, and saturated sodium chloride solution, dried over magnesium sulfate, filtered and concentrated in vacuo to give 196 mg (75% yield) of tert-butyl {2-oxo-2-[7-(3-{[3-(trifluoromethyl)benzoyl]amino}phenyl)pyrazolo[1,5-a]pyrimidin-4(5H)-yl]ethyl}carbamate. MS: (ESI) m/z: 542.3 (M+H)⁺

### EXAMPLE 15

### N-[3-(4-Glycyl-4,5-dihydropyrazolo[1,5-a]pyrimidin-7-yl)phenyl]-3-(trifluoromethyl)benzamide

To a stirred solution of 177 mg (0.3 mmol) of tert-butyl {2-oxo-2-[7-(3-{[3-(trifluoromethyl)benzoyl]amino}phenyl)pyrazolo[1,5-a]pyrimidin-4(5H)-yl]ethyl}carbamate, the product of Example 14, in 6 ml of dichloromethane at 0° C. was added 0.8 ml HCl in dioxane (4M). The solution was stirred at room temperature for 2 hr. The resulting white solid was collected by filtration and washed with anhydrous ethyl ether to give 207 mg (87% yield) of the N-[3-(4-glycyl-4,5-dihydropyrazolo[1,5-a]pyrimidin-7-yl)phenyl]-3-(trifluoromethyl)benzamide. MS (ESI) m/z: 442.2 (M+H)⁺

### EXAMPLE 16

### N-{3-[4-(N-Acetylglycyl)-4,5-dihydropyrazolo[1,5-a]pyrimidin-7-yl]phenyl}-3-(trifluoromethyl)benzamide

To a stirred solution of 98 mg (0.2 mmol) of N-[3-(4-glycyl-4,5-dihydropyrazolo[1,5-a]pyrimidin-7-yl)phenyl]-3-(trifluoromethyl)benzamide, the product of Example 15, in 3 ml of DMF at 0° C. was added 40 mg Hunig's base, followed by 25 mg (0.25 mmol) of acetic anhydride. The solution was stirred at room temperature overnight. After removal of the volatile material in vacuo, the residue was diluted with 80 ml of ethyl acetate. The organic phase was washed with water and saturated sodium chloride solution, dried over magnesium sulfate, filtered and concentrated in vacuo. The residue was purified by flash column chromatography on silica gel eluting with ethyl acetate/hexane to give 48 mg (49% yield) of N-{3-[4-(N-acetylglycyl)-4,5-dihydropyrazolo[1,5-a]pyrimidin-7-yl]phenyl}-3-(trifluoromethyl)benzamide. MS (ESI) m/z: 484.3 (M+H)⁺

### EXAMPLE 17

### tert-Butyl {3-oxo-3-[7-(3-{[3-(trifluoromethyl)benzoyl]amino}phenyl)pyrazolo[1,5-a]pyrimidin-4(5H)-yl]propyl}carbamate

Step 1: tert-Butyl {3-[7-(3-nitrophenyl)pyrazolo[1,5-a]pyrimidin-4(5H)-yl]-3-oxopropyl}carbamate to a stirred solution of 295 mg (1.5 mmol) of Boc-□-alanine in 6 ml of DMF at 0° C. was added 812 mg (1.5 mmol) of PyBOP, followed by 0.56 ml of Hunig's base, and 315 mg (1.3 mmol) of 7-(3-nitrophenyl)-4,5-dihydropyrazolo[1,5-a]pyrimidine, the product of Example 14, Step 1. The solution was stirred at 50° C. overnight and then the volatile material was removed in vacuo and 200 ml of ethyl acetate was added. The organic phase was washed with water and brine, dried over magnesium sulfate, filtered and concentrated in vacuo. The residue was purified by flash column chromatography on silica gel eluting with ethyl acetate/hexane to give 275 mg (51% yield) of tert-butyl {3-[7-(3-nitrophenyl)pyrazolo[1,5-a]pyrimidin-4(5H)-yl]-3-oxopropyl}carbamate. MS (ESI) m/z: 414.3 (M+H)⁺

Step 2: tert-Butyl {3-[7-(3-aminophenyl)pyrazolo[1,5-a]pyrimidin-4(5H)-yl]-3-oxopropyl}carbamate to the stirred solution of 248 mg (0.6 mmol) of tert-butyl {3-[7-(3-nitrophenyl)pyrazolo[1,5-a]pyrimidin-4(5H)-yl]-3-oxopropyl}carbamate in 8 ml of ethanol and 3 ml of water was added 289 mg (5.4 mmol) of ammonium chloride and 101 mg (1.8 mmol) of iron powder. The reaction mixture was refluxed for 3 hr. After filtration, the solution was concentrated in vacuo and 150 ml of ethyl acetate was added. The organic phase was washed with water and brine, dried over magnesium sulfate filtered and concentrated in vacuo. The residue was purified by flash column chromatography on silica gel eluting with ethyl acetate/hexane to give 68 mg (33% yield) of tert-butyl {3-[7-(3-aminophenyl)pyrazolo[1,5-a]pyrimidin-4(5H)-yl]-3-oxopropyl}carbamate. MS (ESI) m/z: 384.3 (M+H)⁺

Step 3: tert-Butyl {3-oxo-3-[7-(3-{[3-(trifluoromethyl)benzoyl]amino}phenyl)pyrazolo[1,5-a]pyrimidin-4(5H)-yl]propyl}carbamate to a stirred solution of 65 mg (0.17 mmol) of tert-butyl {3-[7-(3-aminophenyl)pyrazolo[1,5-a]pyrimidin-4(5H)-yl]-3-oxopropyl}carbamate and 20 mgl of pyridine in 4 ml of dichloromethane was added 39 mg (0.19 mmol) of 3-(trifluoromethyl)benzoyl chloride. The solution was stirred at room temperature overnight and then diluted with 120 ml of dichloromethane. The organic phase was washed with 0.1N HCl solution, water and brine, dried over magnesium sulfate, filtered and concentrated in vacuo. The residue was purified by flash column chromatography on silica gel eluting with ethyl acetate/hexane to give 91 mg (98% yield) of tert-butyl {3-oxo-3-[7-(3-{[3-(trifluoromethyl)benzoyl]amino}phenyl)pyrazolo[1,5-a]pyrimidin-4(5H)-yl]propyl}carbamate. MS (ESI) m/z: 556.4 (M+H)⁺

### EXAMPLE 18

### N-[3-(4-β-Alanyl-4,5-dihydropyrazolo[1,5-a]pyrimidin-7-yl)phenyl]-3-(trifluoromethyl)benzamide

To a stirred solution of 85 mg (0.15 mmol) of tert-butyl {2-oxo-2-[7-(3-{[3-(trifluoromethyl)benzoyl]amino}phenyl)pyrazolo[1,5-a]pyrimidin-4(5H)-yl]ethyl}carbamate, the product of Example 17, in 2 ml of dichloromethane at 0° C. was added 0.8 ml HCl in dioxane (4M). The solution was stirred at room temperature for 4 hr and the resulting white solid was collected by filtration, and washed with anhydrous ethyl ether to provide 83 mg (99% yield) of N-[3-(4-β-alanyl-4,5-dihydropyrazolo[1,5-a]pyrimidin-7-yl)phenyl]-3-(trifluoromethyl)benzamide. MS (ESI) m/z: 456.5 (M+H)⁺

### EXAMPLE 19

### N-{3-[4-(N-Acetyl-β-alanyl)-4,5-dihydropyrazolo[1,5-a]pyrimidin-7-yl]phenyl}-3-(trifluoromethyl)benzamide

To a stirred solution of 76 mg (0.15 mmol) of N-[3-(4-β-alanyl-4,5-dihydropyrazolo[1,5-a]pyrimidin-7-yl)phenyl]-3-(trifluoromethyl)benzamide, the product of Example 18, in 3 ml of DMF at 0° C. was added 50 mg of Hunig's base, followed by 19 mg (0.19 mmol) of acetic anhydride. The solution was stirred at room temperature overnight. After removal of the volatile material in vacuo, 80 ml of ethyl acetate was added. The organic phase was washed with water and brine, dried over magnesium sulfate, filtered and concentrated in vacuo. The residue was purified by flash column chromatography on silica gel eluting with ethyl acetate/hexane to provide 39 mg (53% yield) of N-{3-[4-(N-acetyl-b-alanyl)-4,5-dihydropyrazolo[1,5-a]pyrimidin-7-yl]phenyl}-3-(trifluoromethyl)benzamide. MS (ESI) m/z: 498.3 (M+H)⁺

### EXAMPLE 20

### N-[4-Chloro-3-(4,5-dihydropyrazolo[1,5-a]pyrimidin-7-yl)phenyl]-3-(trifluoromethyl)benzamide

According to the procedure of Example 1, Steps 1-3, 5′-amino-2′-chloroacetophenone provides N-(4-chloro-3-pyrazolo[1,5-a]pyrimidin-7-ylphenyl)-3-(trifluoromethyl)benzamide. To a suspension of 501 mg (1.2 mmol) of N-(4-chloro-3-pyrazolo[1,5-a]pyrimidin-7-ylphenyl)-3-(trifluoromethyl)benzamide in 8 ml of acetic acid was added, in portions, 166 mg (2.6 mmol) of sodium cyanoborohydride at room temperature under nitrogen. The mixture was stirred at room temperature for 2 hr. After removal of the volatile material in vacuo, 200 ml of ethyl acetate was added. The organic phase was neutralized with a saturated sodium bicarbonate solution, washed with brine, dried over magnesium sulfate, filtered and concentrated in vacuo to give 500 mg (99% yield) of N-[4-chloro-3-(4,5-dihydropyrazolo[1,5-a]pyrimidin-7-yl)phenyl]-3-(trifluoromethyl)benzamide. MS (ESI) m/z: 419.2 (M+H)⁺

What is claimed is:

1. A compound of Formula (I): 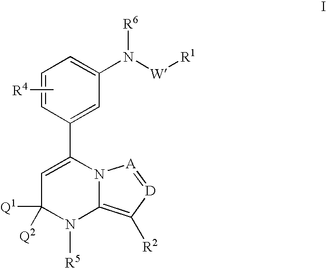
or a pharmaceutically acceptable salt thereof,
wherein
A is C—R³or N;
D is N or C—R³ provided when D is N, A is C—R³;
R¹ is phenyl, naphthyl, or a 5-10 membered mono- or bicyclic heteroaryl ring containing 1-3 heteroatoms selected from N, O or S, wherein the phenyl, naphthyl and heteroaryl rings are optionally substituted with one to four substituents selected from the group consisting of -J, —NO₂, —CN, —N₃, —CHO, —CF₃, —OCF₃, —R⁷, —OR⁷, —S(O)ₘR⁷, —NR⁷R¹², —NR¹³S(O)ₘR⁷, —OR⁹OR⁷, —OR⁹NR⁷R¹², —N(R¹¹)R⁹OR⁷, —N(R¹³)R⁹NR⁷R¹⁴, —NR¹³C(O)R⁷, —C(O)R⁷, —C(O)OR⁷, —C(O)NR⁷R¹², —OC(O)R⁷, —OC(O)OR⁷, —OC(O)NR⁷R¹², NR¹¹C(O)R⁷, —NR¹¹C(O)OR⁷, —NR¹¹C(O)NR⁷R¹², —R⁸OR⁷, —R⁸NR⁷R¹², —R⁸S(O)ₘR⁷, —R⁸C(O)R⁷, —R⁸C(O)OR⁷, —R⁸C(O)NR⁷R¹², —R⁸OC(O)R⁷, —R⁸OC(O)OR⁷, —R⁸OC(O)NR⁷R¹², —R⁸NR¹¹C(O)R⁷, —R⁸NR¹¹C(O)OR⁷, —R⁸NR¹¹C(O)NR⁷R¹², —YR⁸R¹⁰, —YR⁸NR⁷R¹² or —YR¹⁰.
R² and R³ are independently selected from R⁷, J, —C(O)OR⁷, —C(O)NR⁷R¹², —NR⁶C(O)R⁷, —CN, 5-7 membered heterocyclic ring, 5-10 membered heteroaryl ring containing 1-3 heteroatoms selected from N, O or S, or aryl ring, wherein the R⁷ groups, heterocyclic, heteroaryl and aryl rings can be optionally substituted with one to four substituents selected from the group consisting of -J, —NO₂, —CN, —N₃, —CHO, —CF₃, —OCF₃, —R¹³, —OR¹³, —S(O)ₘR¹³, —NR¹³R¹⁴, —NR¹¹S(O)ₘR¹³, —OR⁹OR¹³, —OR⁹NR¹³R¹², —N(R¹¹)R⁹OR¹³, —N(R¹¹)R⁹NR¹³R¹², —NR¹¹C(O)R¹³, —C(O)R¹³, —C(O)OR¹³, —C(O)NR¹³R¹², —OC(O)R¹³, —OC(O)OR¹³, —OC(O)NR¹³R¹², NR¹¹C(O)R¹³, —NR¹¹C(O)OR¹³, —NR¹¹C(O)NR¹³R¹², —R⁸OR¹³, —R⁸NR¹³R¹², —R⁸S(O)ₘR¹³, —R⁸C(O)R¹³, —R⁸C(O)OR¹³, —R⁸C(O)NR¹³R¹², —R⁸OC(O)R¹³, —R⁸OC(O)OR¹³, —R⁸OC(O)NR¹³R¹², —R⁸NR¹¹C(O)R¹³, —R⁸NR¹¹C(O)OR¹³ or —R⁸NR¹¹C(O)NR¹³R¹² or YR¹⁰;
R⁴ is H, -J, —NO₂, —CN, —N₃, —CHO, —CF₃, —OCF₃, —R⁷, —OR⁷, —S(O)ₘR⁷, —NR⁷R¹², —NR¹¹S(O)ₘR⁷, —OR⁹OR, —OR⁹NR⁷R¹², —N(R¹¹)R⁹OR⁷, —N(R¹¹)R⁹NR⁷R¹², —NR¹¹C(O)R⁷, —C(O)R⁷, —C(O)OR⁷, —C(O)NR⁷R¹², —OC(O)R⁷, —OC(O)OR⁷, —OC(O)NR⁷R¹², NR¹¹C(O)R⁷, —NR¹¹C(O)OR⁷, —NR¹¹C(O)NR⁷R¹², —R⁸OR⁷, —R⁸NR⁷R¹², —R⁸S(O)ₘR⁷, —R⁸C(O)R⁷, —R⁸C(O)OR⁷, —R⁸C(O)NR⁷R¹², —R⁸OC(O)OR⁷, —R⁸OC(O)NR⁷R¹², —R⁸NR¹¹C(O)R⁷, —R⁸NR¹¹C(O)OR⁷ or —R⁸NR¹¹C(O)NR⁷R¹² or YR¹⁰;
R⁵ is H, —CHO, —R⁷, —S(O)ₘR⁷, —C(O)R⁷, —C(O)OR⁷, —C(O)NR⁷R¹², —R⁸OR⁷, —R⁸NR⁷R¹², —R⁸S(O)ₘR⁷, —R⁸C(O)R⁷, —R⁸C(O)OR⁷, —R⁸C(O)NR⁷R¹², —R⁸OC(O)R⁷, —R⁸OC(O)OR⁷, —R⁸OC(O)NR⁷R¹², —R⁸NR¹¹C(O)R⁷, —R⁸NR¹¹C(O)OR⁷ or —R⁸NR¹¹C(O)NR⁷R¹² or YR¹⁰;
R⁶ is H;
R⁷ is H, alkyl of 1-6 carbon atoms, branched alkyl of 1-8 carbon atoms, cis-alkenyl of 2-6 carbon atoms, a trans-alkenyl of 2-6 carbon atoms, or an alkynyl of 2-6 carbon atoms, each said alkyl, alkenyl, and alkynyl being optionally substituted with -J, —NO₂, —CN, —N₃, —CHO, —CF₃, —OCF₃, —R¹³, —OR¹³, —S(O)ₘR¹³, —NR¹³R¹⁴, —NR¹³S(O)ₘR¹⁴, —OR⁹OR¹³, —OR⁹NR¹³R¹⁴, —N(R¹³)R⁹OR¹⁴, —N(R¹³)R⁹NR¹⁴R¹⁵, —NR¹³C(O)R¹⁴, —C(O)R¹³, —C(O)OR¹³, —C(O)NR¹³R¹⁴, —OC(O)R¹³, —OC(O)OR¹³, —OC(O)NR¹³R¹⁴, NR¹³C(O)R¹⁴, —NR¹³C(O)OR¹⁴, —NR¹³C(O)NR¹⁴R¹⁵, —R⁸OR¹³, —R⁸NR¹³R¹⁴, —R⁸S(O)ₘR¹³, —R⁸C(O)OR¹³, —R⁸C(O)NR¹³R¹⁴, —R⁸OC(O)R¹³, —R⁸OC(O)OR¹³, —R⁸OC(O)NR¹³R¹⁴, —R⁸NR¹³C(O)R¹⁴, —R⁸NR¹³C(O)OR¹⁴, —R⁸NR¹³C(O)NR¹⁴R¹⁵ or —YR¹⁰;
R⁸ is a divalent group comprising alkyl of 1-6 carbon atoms, alkenyl of 2-6 carbon atoms, and alkynyl of 2-6 carbon atoms;
R⁹ is a divalent alkyl group of 2-6 carbon atoms;
R¹⁰ is a cycloalkyl ring of 3-10 carbons; or a bicycloalkyl ring of 3-10 carbons; or aryl; or heterocyclic or a heteroaryl ring containing 1-3 heteroatoms selected from N, O or S; or a heteroaryl fused to one to three aryl or heteroaryl rings; wherein any of the aryl, cycloalkyl, bicycloalkyl, heterocyclic or heteroaryl rings may be optionally substituted with one to four substituents selected from the group consisting of —H, -aryl, —CH₂-aryl, —NH-aryl, —O-aryl, —S(O)ₘ-aryl, -J, —NO₂, —CN, —N₃, —CHO, —CF₃, —OCF₃, —R⁷, —OR⁷, —S(O)ₘR⁷, —NR⁷R¹², —NR¹¹S(O)ₘR⁷, —OR⁹OR⁷, —OR⁹NR⁷R¹², —N(R¹¹)R⁹OR⁷, —N(R¹¹)R⁹NR⁷R¹², —NR¹¹C(O)R⁷, —C(O)R⁷, —C(O)OR⁷, —C(O)NR⁷R¹², —OC(O)R⁷—, —OC(O)OR⁷, —OC(O)NR⁷R¹², —NR¹¹C(O)R⁷, —NR¹¹C(O)OR⁷, —NR¹¹C(O)NR⁷R¹², —R⁸OR⁷, R⁸NR⁷R¹², —R⁸S(O)ₘR⁷, —R⁸C(O)R⁷, —R⁸C(O)OR⁷, —R⁸C(O)NR⁷R¹², —R⁸C(O)R⁷, —R⁸C(O)OR⁷, —R⁸C(O)NR⁷R¹², —R⁸OC(O)R⁷, —R⁸OC(O)OR⁷, —R⁸OC(O)NR⁷R¹², —R⁸NR¹¹C(O)R⁷, —R⁸NR¹¹C(O)OR⁷, or —R⁸NR¹¹C(O)NR⁷R¹²;
R¹¹ is H, alkyl of 1-6 carbon atoms, branched alkyl of 1-8 carbon atoms, cis-alkenyl of 2-6 carbon atoms, a trans-alkenyl of 2-6 carbon atoms, or an alkynyl of 2-6 carbon atoms;
R¹² is H, alkyl of 1-6 carbon atoms, branched alkyl of 1-8 carbon atoms, cis-alkenyl of 2-6 carbon atoms, a trans-alkenyl of 2-6 carbon atoms, or an alkynyl of 2-6 carbon atoms;
R⁷ and R¹² together with the N to which they are attached may join to form a 3 to 8 membered ring;
R¹³, R¹⁴, and R¹⁵ are independently selected from the group of H, alkyl of 1-6 carbon atoms, branched alkyl of 1-8 carbon atoms, cis-alkenyl of 2-6 carbon atoms, a trans-alkenyl of 2-6 carbon atoms, and an alkynyl of 2-6 carbon atoms;
J is fluoro, chloro, bromo, and iodo;
m is an integer of 0-2;
W′ is —C(O);
Y is a bond, a divalent alkyl group of 1-6 carbon atoms, —NH—, —O—, —NR⁷—, —C═C—, cis- CH═CH—, or trans- —CH═CH—; and
Q¹ and Q² are hydrogen or when taken together with the carbon to which they are attached may form a carbonyl (CαO) group.

2. A composition comprising an effective amount of a compound or claim 1 or a pharmaceutically acceptable salt of the compound of claim 1 and a physiologically acceptable vehicle.

3. A method of treating, inhibiting the growth of, or eradicating neoplasms in a mammal in need thereof which comprises providing to said mammal an effective amount of a compound of claim 1 or a composition of claim 2.

4. The method of treating cancer in a mammal in need thereof which comprises providing to said mammal an effective amount of a compound of claim 1 or a composition of claim 2.

5. The method of claim 4 wherein the cancer is selected from the group consisting of breast, kidney, bladder, mouth, larynx, esophagus, stomach, colon, ovary, lung, pancreas, skin, liver, prostate and brain.

6. The method of claim 4, wherein the mammal is human.

7. The compound of claim 1, wherein the compound is: N-[3-(4,5-dihydropyrazolo[1,5-a]pyrimidin-7-yl)phenyl]-3-(trifluoromethyl)benzamide.

8. The compound of claim 1, wherein the compound is: N-{3-[4-(methylsulfonyl)-4,5-dihydropyrazolo[1,5-a]pyrimidin-7-yl]phenyl}-3-(trifluoromethyl)benzamide or a pharmaceutically acceptable salt thereof.

9. The compound of claim 1, wherein the compound is: methyl 7-(3-{[3-(trifluoromethyl)benzoyl]amino}phenyl)pyrazolo[1,5-a]pyrimidine-4(5H)-carboxylate or a pharmaceutically acceptable salt thereof.

10. The compound of claim 1, wherein the compound is: ethyl 7-(3-{[3-(trifluoromethyl)benzoyl]amino}phenyl)pyrazolo[1,5-a]pyrimidine-4(5H)-carboxylate or a pharmaceutically acceptable salt thereof.

11. The compound of claim 1, wherein the compound is: N-isopropyl-7-(3-{[3-(trifluoromethyl)benzoyl]amino}phenyl)pyrazolo[1,5-a]pyrimidine-4(5H)-carboxamide or a pharmaceutically acceptable salt thereof.

12. The compound of claim 1, wherein the compound is: tert-butyl 7-(3-{[3-(trifluoromethyl)benzoyl]amino}phenyl)pyrazolo[1,5-a]pyrimidine-4(5H)-carboxylate or a pharmaceutically acceptable salt thereof.

13. The compound of claim 1, wherein the compound is: N-[3-(4-but-2-yn-1-yl-4,5-dihydropyrazolo[1,5-a]pyrimidin-7-yl)phenyl]-3-(trifluoromethyl)benzamide or a pharmaceutically acceptable salt thereof.

14. The compound of claim 1, wherein the compound is: ethyl 7-(3-{[3-(trifluoromethyl)benzoyl]amino}phenyl)-4,5-dihydropyrazolo[1,5-a]pyrimidine-3-carboxylate or a pharmaceutically acceptable salt thereof.

15. The compound of claim 1, wherein the compound is: N-ethyl-7-(3-{[3-(trifluoromethyl)benzoyl]amino}phenyl)pyrazolo[1,5-a]pyrimidine-4(5H)-carboxamide or a pharmaceutically acceptable salt thereof.

16. The compound of claim 1, wherein the compound is: N-[3-(4-acetyl-4,5-dihydropyrazolo[1,5-a]pyrimidin-7-yl)phenyl]-3-(trifluoromethyl)benzamide or a pharmaceutically acceptable salt thereof.

17. The compound of claim 1, wherein the compound is: ethyl 4-acetyl-7-(3-{[3-(trifluoromethyl)benzoyl]amino}phenyl)-4,5-dihydropyrazolo[1,5-a]pyrimidine-3-carboxylate or a pharmaceutically acceptable salt thereof.

18. The compound of claim 1, wherein the compound is: tert-butyl 7-(3-{(tert-butoxycarbonyl)[3-(trifluoromethyl)benzoyl]amino}phenyl)pyrazolo[1,5-a]pyrimidine-4(5H)-carboxylate 4(5H)-carboxylate or a pharmaceutically acceptable salt thereof.

19. The compound of claim 1, wherein the compound is: 3-ethyl 4-methyl 7-(3-{[3-(trifluoromethyl)benzoyl]amino}phenyl)pyrazolo[1,5-a]pyrimidine-3,4(5H)-dicarboxylate or a pharmaceutically acceptable salt thereof.

20. The compound of claim 1, wherein the compound is: tert-butyl {2-oxo-2-[7-(3-{[3-(trifluoromethyl)benzoyl]amino}phenyl)pyrazolo[1,5-a]pyrimidin-4(5H)-yl]ethyl}carbamate or a pharmaceutically acceptable salt thereof.

21. The compound of claim 1, wherein the compound is: N-[3-(4-glycyl-4,5-dihydropyrazolo[1,5-a]pyrimidin-7-yl)phenyl]-3-(trifluoromethyl)benzamide or a pharmaceutically acceptable salt thereof.

22. The compound of claim 1, wherein the compound is: N-{3-[4-(N-acetylglycyl)-4,5-dihydropyrazolo[1,5-a]pyrimidin-7-yl]phenyl}-3-(trifluoromethyl)benzamide or a pharmaceutically acceptable salt thereof.

23. The compound of claim 1, wherein the compound is: tert-butyl {3-oxo-3-[7-(3-{[3-(trifluoromethyl)benzoyl]amino}phenyl)pyrazolo[1,5-a]pyrimidin-4(5H)-yl]propyl}carbamate or a pharmaceutically acceptable salt thereof.

24. The compound of claim 1, wherein the compound is: N-[3-(4-β-alanyl-4,5-dihydropyrazolo[1,5-a]pyrimidin-7-yl)phenyl]-3-(trifluoromethyl)benzamide or a pharmaceutically acceptable salt thereof.

25. The compound of claim 1, wherein the compound is: N-{3-[4-(N-acetyl-β-alanyl)-4,5-dihydropyrazolo[1,5-a]pyrimidin-7-yl]phenyl}-3-(trifluoromethyl)benzamide or a pharmaceutically acceptable salt thereof.

26. The compound of claim 1, wherein the compound is: N-[4-chloro-3-(4,5-dihydropyrazolo[1,5-a]pyrimidin-7-yl)phenyl]-3-(trifluoromethyl)benzamide or a pharmaceutically acceptable salt thereof.
